(19) United States
(12) Patent Application Publication
(10) Pub. No.: US 2023/0381174 A1
(43) Pub. Date: Nov. 30, 2023

(54) THIONOESTER-DERIVATIVE OF RABEXIMOD FOR THE TREATMENT OF INFLAMMATORY AND AUTOIMMUNE DISORDERS

(71) Applicant: Vironova Thionationl AB, STOCKHOLM (SE)

(72) Inventors: Jan BERGMAN, SPÅNGA (SE); Robert BERG, RÖNNINGE (SE); Ngarita KINGI, HÄGERSTEN (SE); Eva O'KEEFFE, STOCKHOLM (SE)

(21) Appl. No.: 18/245,976

(22) PCT Filed: Sep. 21, 2021
(86) PCT No.: PCT/EP2021/075944

§ 371 Date: Mar. 20, 2023

(30) Foreign Application Priority Data
Sep. 21, 2020 (EP) 20197307.0

(51) Int. Cl. A61K 31/4985; A61P 19/02; A61P 37/06

(52) U.S. Cl. CPC A61K 31/4985; A61P 19/02; A61P 37/06

(57) ABSTRACT

The compound 2-(9-chloro-2,3-dimethyl-6H-indolo[2,3-b]quinoxalin-6-yl)-N-(2-(dimethylamino)ethyl)ethanethioamide, for use in the treatment of inflammation, an inflammatory disorder or an autoimmune disorder.

### FIELD OF THE INVENTION

The present invention relates to a derivative of the compound Rabeximod (2-(9-chloro-2,3-dimethyl-6H-indolo[2,3-b]quinoxalin-6-yl)-N-(2-(dimethylamino)ethyl)acetamide) for the treatment of a condition selected from inflammation, an inflammatory disorder and an autoimmune disorder.

### BACKGROUND OF THE INVENTION

Rabeximod was described in international patent application No. PCT/SE2005/000718, published as WO 2005/123741 A1 as therapeutically active in autoimmune disorders such as multiple sclerosis (MS) and rheumatoid arthritis (RA). The compound is presently in clinical phase as a drug for the treatment of RA, with promising results. However, there is still a need for new drugs for the treatment of various autoimmune disorders, such as RA.

WO 2014/140321 A1 describes thionated Rabeximod, i.e. 2-(9-chloro-2,3-dimethyl-6H-indolo[2,3-b]quinoxalin-6-yl)-N-(2-(dimethylamino)ethyl)ethanethioamide, and a method for its preparation, as well as the use of the compound as an antiviral agent.

### SUMMARY OF THE INVENTION

A first aspect is the compound 2-(9-chloro-2,3-dimethyl-6H-indolo[2,3-b]quinoxalin-6-yl)-N-(2-(dimethylamino)ethyl)ethanethioamide or a pharmaceutically acceptable salt thereof, for use in the treatment of a condition selected from inflammation, and inflammatory disorder or an autoimmune disorder.

A further aspect is the use of 2-(9-chloro-2,3-dimethyl-6H-indolo[2,3-b]quinoxalin-6-yl)-N-(2-(dimethylamino)ethyl)ethanethioamide or a pharmaceutically acceptable salt thereof, in the manufacture of a medicament for the treatment of a condition selected from inflammation, and inflammatory disorder or an autoimmune disorder.

A further aspect is the use of 2-(9-chloro-2,3-dimethyl-6H-indolo[2,3-b]quinoxalin-6-yl)-N-(2-(dimethylamino)ethyl)ethanethioamide or a pharmaceutically acceptable salt thereof, in the treatment of a condition selected from inflammation, and inflammatory disorder or an autoimmune disorder.

A further aspect is a method for the treatment of a condition selected from inflammation, and inflammatory disorder or an autoimmune disorder, by administering 2-(9-chloro-2,3-dimethyl-6H-indolo[2,3-b]quinoxalin-6-yl)-N-(2-(dimethylamino)ethyl)ethanethioamide or a pharmaceutically acceptable salt thereof, to a mammal in need of such treatment.

A further aspect is a pharmaceutical composition comprising the compound 2-(9-chloro-2,3-dimethyl-6H-indolo[2,3-b]quinoxalin-6-yl)-N-(2-(dimethylamino)ethyl)ethanethioamide or a pharmaceutically acceptable salt thereof, for use in the treatment of a condition selected from inflammation, and inflammatory disorder or an autoimmune disorder.

A further aspect is the compound 2-(9-chloro-2,3-dimethyl-6H-indolo[2,3-b]quinoxalin-6-yl)-N-(2-(dimethylamino)ethyl)ethanethioamide or a pharmaceutically acceptable salt thereof, or a pharmaceutical composition comprising said compound or salt and optionally a pharmaceutically acceptable excipient, for use in the treatment of rheumatoid arthritis.

A further aspect is the compound 2-(9-chloro-2,3-dimethyl-6H-indolo[2,3-b]quinoxalin-6-yl)-N-(2-(dimethylamino)ethyl)ethanethioamide or a pharmaceutically acceptable salt thereof, or a pharmaceutical composition comprising said compound or salt and optionally a pharmaceutically acceptable excipient, for use in the treatment of multiple sclerosis.

A further aspect is the compound 2-(9-chloro-2,3-dimethyl-6H-indolo[2,3-b]quinoxalin-6-yl)-N-(2-(dimethylamino)ethyl)ethanethioamide or a pharmaceutically acceptable salt thereof, or a pharmaceutical composition comprising said compound or salt and optionally a pharmaceutically acceptable excipient, for use in the treatment of a condition wherein reactive oxygen species (ROS) play a pathogenic role.

Further aspects of the invention and embodiments thereof are described herein below.

### BRIEF DESCRIPTION OF THE DRAWINGS

FIG. 1 is a graph showing the Reactive Oxygen Species (ROS) production, measured with Relative Luminescence unit (RLU), in PBMCs activated with PMA at 30 ng/ml, with or without treatment with the inventive compound at different concentrations. Results are presented as mean value of triplicate samples+/−SEM.

FIG. 2 is a graph showing the Reactive Oxygen Species (ROS) production, measured with Relative Luminescence unit (RLU), in PBMCs activated with PMA at 30 ng/ml, with or without treatment with Rabeximod at different concentrations. Results are presented as mean value of triplicate samples+/−SEM.

FIG. 3 is a graph showing the concentration of the TNF-α in whole blood after stimulation with LPS at 500 ng/ml and treatment with either the inventive compound (I) or Rabeximod (R) at concentrations ranging from 312.5 nM to 5000 nM. Results are presented as mean value of two replicates+/−SEM.

FIG. 4 is a graph showing the concentration of IL-6 in whole blood after stimulation with LPS at 500 ng/ml and treatment with either the inventive compound (I) or Rabeximod (R) at concentrations ranging from 312.5 nM to 5000 nM. Results are presented as mean value of two replicates+/−SEM.

FIG. 5 is a graph showing the concentration of IL-1β in whole blood after stimulation with LPS at 500 ng/ml and treatment with either the inventive compound (I) or Rabeximod (R) at concentrations ranging from 312.5 nM to 5000 nM. Results are presented as mean value of two replicates+/−SEM.

FIG. 6 is a graph representing the mean arthritis severity in the collagen antibody induced arthritis (CAIA) model in mice treated with κ mg/kg, 10 mg/kg or 20 mg/kg of the inventive compound at 5, 7, 9, 11, 13 and 15 days after antibody injection, or with vehicle only, and using a macroscopic scoring system of the limbs of the mice, with a maximum arthritic score of 60. (8 animals per group)

FIG. 7 is a graph representing the mean arthritis severity in the collagen antibody induced arthritis (CAIA) model in mice treated with κ mg/kg, 10 mg/kg or 20 mg/kg of Rabeximod at 7, 9, 11, 13 and 15 days after antibody injection, or with vehicle only, and using a macroscopic scoring system of the four limbs of the mice, with a maximum arthritic score of (8 animals per group)

FIG. 8 is a graph representing the immunohistochemical arthritic score of front paws of mice in the CAIA model, treated with vehicle only or with 20 mg/kg of either the inventive compound or Rabeximod, with a maximum arthritic score of 3.

FIG. 9 is a graph representing the mean arthritis severity in the pristane-induced arthritis (PIA) model in rat treated with 20 mg/kg of either Rabeximod or the inventive compound at 5, 7, 9, 11, 13 and 15 days after disease induction, or with vehicle only, and using a macroscopic scoring system of the limbs of the mice, with a maximum arthritic score of 60. (10 animals per group)

### DETAILED DESCRIPTION OF THE INVENTION

The term “autoimmune disorder” refers to any disorder arising from an inappropriate immune response of the body against substances and tissues normally present in the body (autoimmunity). Such response may be restricted to certain organs or involve a particular tissue in different places. Exemplary autoimmune disorders are acute disseminated encephalomyelitis (ADEM), Addison's disease, agammaglobulinemia, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease/Balo concentric sclerosis, Beh [symbol] et's disease, Berger's disease, Bickerstaff s encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, CREST syndrome, Crohn's disease (one of two types of idiopathic inflammatory bowel disease “IBD”), Cushing's Syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, epidermolysis bullosa acquisita, erythema nodosum, erythroblastosis fetalis, essential mixed cryoglobulinemia, Evan's syndrome, fibrodysplasia ossificans progressive, fibrosing alveolitis (or Idiopathic pulmonary fibrosis), gastritis, gastrointestinal pemphigoid, glomerulonephritis, Goodpasture's syndrome, Graves' disease, Guillain-Barré syndrome (GBS), Hashimoto's encephalopathy, Hashimoto's thyroiditis, Henoch-Schonlein purpura, herpes gestationis (aka gestational pemphigoid), Hidradenitis suppurativa, Hughes-Stovin syndrome, hypogammaglobulinemia, idiopathic inflammatory demyelinating diseases, idiopathic pulmonary fibrosis, idiopathic thrombocytopenic purpura, IgA nephropathy, inclusion body myositis, chronic inflammatory demyelinating polyneuropathy, interstitial cystitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, Majeed syndrome, Meniere's disease, microscopic polyangiitis, mixed connective tissue disease, morphea, Mucha-Habermann disease (aka pityriasis lichenoides et varioliformis acuta), multiple sclerosis, myasthenia gravis, myositis, narcolepsy, neuromyelitis optica (also Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonage-Turner syndrome, pars planitis, pemphigus vulgaris, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatic fever, sarcoidosis, schizophrenia, Schmidt syndrome another form of APS, Schnitzler syndrome, Scleritis, Scleroderma, Serum Sickness, Sjögren's syndrome, spondyloarthropathy, stiff person syndrome, subacute bacterial endocarditis (SBE), Susac's syndrome, Sweet's syndrome, sympathetic ophthalmia, systemic lupus erythematosis, Takayasu's arteritis, temporal arteritis (also known as “giant cell arteritis”), thrombocytopenia, Tolosa-Hunt syndrome, transverse myelitis, ulcerative colitis (one of two types of idiopathic inflammatory bowel disease “IBD”), undifferentiated connective tissue disease different from mixed connective tissue disease, undifferentiated spondyloarthropathy, urticarial vasculitis, vasculitis, vitiligo, and Wegener's granulomatosis. The term “inflammation” as used herein may refer to acute inflammation (including hyperinflammation) and chronic inflammation.

Chronic inflammation may follow an ineffective acute inflammatory response or with no clinically apparent acute phase. Chronic inflammation is distinguished from acute inflammation by a general absence of the cardinal signs of inflammation (redness, swelling, pain, and increased temperature), and of being of a duration sufficient to permit the tissue manifestations of the immune response and repair. Chronic inflammation often is accompanied by extensive tissue necrosis and tissue repair by fibrosis.

The term “inflammatory disorder” refers to a pathological state associated with inflammation, typically caused by leukocyte infiltration. The inflammatory disorder may be acute or chronic. Exemplary inflammatory disorders include inflammatory skin diseases, including, without limitation, psoriasis and atopic dermatitis, systemic scleroderma and sclerosis, responses associated with inflammatory bowel disease (IBD) (such as Crohn's disease and ulcerative colitis), ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms, cerebral edema secondary to stroke, cranial trauma, hypovolemic shock, asphyxia, adult respiratory distress syndrome, acute-lung injury, Behcet's Disease, dermatomyositis, polymyositis, multiple sclerosis (MS), dermatitis, meningitis, encephalitis, uveitis, osteoarthritis, lupus nephritis, autoimmune diseases such as rheumatoid arthritis (RA), Sjögren's syndrome, vasculitis, diseases involving leukocyte diapedesis, central nervous system (CNS) inflammatory disorder, multiple organ injury syndrome secondary to septicemia or trauma, alcoholic hepatitis, bacterial pneumonia, antigen-antibody complex mediated diseases including glomerulonephritis, sepsis, sarcoidosis, immunopathologic responses to tissue or organ transplantation, inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis, etc.

The term inflammatory disorder also refers to a disorder caused by or associated with hyperinflammation, e.g. a hyperinflammatory syndrome.

The term “hyperinflammatory syndrome” includes a syndrome selected from immunodeficiency-associated hyperinflammatory syndromes, macrophage activation syndrome, infection-associated syndrome (e.g. associated with covid-19), malignancy-associated hyperinflammatory syndrome.

“Therapeutically effective amount” means an amount of a compound that, when administered to a subject for treating a disease state, is sufficient to achieve a therapeutic effect in the treatment for the disease state. The “therapeutically effective amount” will vary depending on the compound, the disease state being treated, the severity of the disease treated, the age and relative health of the subject, the route and form of administration, the judgment of the attending medical or veterinary practitioner, etc. The therapeutic effect may be observed in a manner that is objective (i.e. measurable by some test or marker) or subjective (i.e. the subject gives an indication of and/or feels an effect).

The term “excipient” refers to pharmaceutically acceptable chemicals, such as known to those of ordinary skill in the art of pharmacy to aid the administration of the medicinal agent. It is a compound that is useful in preparing a pharmaceutical composition, generally safe, non-toxic and neither biologically nor otherwise undesirable, and includes excipients that are acceptable for veterinary use as well as human pharmaceutical use. Exemplary excipients include binders, surfactants, diluents, disintegrants, antiadherents, and lubricants.

“Pharmaceutically acceptable” means being useful in preparing a pharmaceutical composition that is generally safe, non-toxic and includes being useful for veterinary use as well as human pharmaceutical use.

The compound for use according to the present invention, viz. 2-(9-chloro-2,3-dimethyl-6H-indolo[2,3-b]quinoxalin-6-yl)-N-(2-(dimethylamino)ethyl)ethanethioamide, is herein also referred to as the “compound of the invention” or “inventive compound”. The compound is represented by structural formula (I)

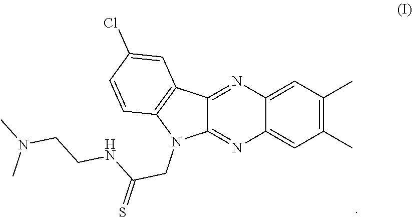

The inventive compound is useful in the treatment of a condition as defined herein above. In some embodiments, the condition is inflammation or an inflammatory disorder, e.g. chronic inflammation or a chronic inflammatory disorder. In some embodiments, the condition is inflammation, e.g. chronic inflammation. In some embodiments, the condition is an acute inflammation. In some embodiments, the condition is hyperinflammation. In some embodiments, the condition is an inflammatory disorder, e.g. a chronic inflammatory disorder. In some embodiments, the condition is a hyperinflammatory syndrome, e.g. an infection-associated hyperinflammatory syndrome, such as hyperinflammatory syndrome in connection with a respiratory disease, e.g. pneumonia or covid-19, in particular covid-19. In some embodiments, the condition is an autoimmune disorder. In some particular embodiments, the condition is rheumatoid arthritis. In some further particular embodiments, the condition is multiple sclerosis.

It is noted that rheumatoid arthritis and multiple sclerosis may each be classified as both an inflammatory disorder and an autoimmune disorder.

Consequently, one aspect of the invention is a pharmaceutical composition comprising a therapeutically effective amount of 2-(9-chloro-2,3-dimethyl-6H-indolo[2,3-b]quinoxalin-6-yl)-N-(2-(dimethylamino)ethyl)ethanethioamide, or pharmaceutically acceptable salt thereof, and optionally a pharmaceutically acceptable excipient, for use in the treatment of a condition, as mentioned herein.

The intended pharmaceutical use is for the treatment of a mammal, preferably a human, but also an animal such as a pet animal, e.g. a dog or a cat, or a farm animal, or a laboratory animal, or e.g. a horse. In some embodiments, the mammal is a human, e.g. a human suffering from rheumatoid arthritis. In some embodiments, the mammal is a human suffering from multiple sclerosis. In some other embodiments, the mammal is a pet animal, e.g. a dog (the inflammatory or autoimmune disorder in a dog may be e.g. rheumatoid arthritis, granulomatous meningoencephalomyelitis, or degenerative myelopathy). Thus, in some embodiments, the mammal is a dog suffering from a rheumatoid arthritis. In some other embodiments, the mammal is a dog suffering from granulomatous meningoencephalomyelitis.

In some further embodiments, the mammal is a horse, e.g. a horse suffering from rheumatoid arthritis. In the embodiment wherein the mammal is an animal, expressions such as “pharmaceutically acceptable” etc. should be understood as referring to acceptable for the corresponding veterinary use, etc.

Herein, the expression “compound of the present invention” or “compound of the invention” or “inventive compound” should be construed as referring to 2-(9-chloro-2,3-dimethyl-6H-indolo[2,3-b]quinoxalin-6-yl)-N-(2-(dimethylamino)ethyl)ethanethioamide, but also as referring to a pharmaceutically acceptable salt of said compound, unless otherwise indicated or apparent from the context. In some embodiments, the compound is used in the form of the “free base”, i.e. the non-salt form.

In some embodiments, the compound of the invention may be provided in the form of a pharmaceutically acceptable salt. Acid addition salts of the inventive compound may in a manner known per se be transformed into the free base using basic agents such as alkali or by ion exchange. The free base obtained may also form salts with organic or inorganic acids. Alkali addition salts of the inventive compound may in a manner known per se be transformed into the free acid by using acidic agents such as acid or by ion exchange. The free acid obtained may also form salts with organic or inorganic bases.

In the preparation of acid or base addition salts, preferably such acids or bases are used which form suitably therapeutically acceptable salts. Examples of such acids are hydrohalogen acids, sulfuric acid, phosphoric acid, nitric acid, aliphatic, alicyclic, aromatic or heterocyclic carboxylic or sulfonic acids, such as formic acid, acetic acid, propionic acid, succinic acid, glycolic acid, lactic acid, malic acid, tartaric acid, citric acid, ascorbic acid, maleic acid, hydroxymaleic acid, pyruvic acid, p-hydroxybenzoic acid, embonic acid, methanesulfonic acid, ethanesulfonic acid, hydroxyethanesulfonic acid, halogenbenzenesulfonic acid, toluenesulfonic acid or naphthalenesulfonic acid. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Examples of bases useful in preparing salts of the present invention include sodium hydroxide, potassium hydroxide, ammonium hydroxide, potassium carbonate, and the like.

Pharmaceutical formulations may be prepared by combining the active substance, which in the present case is the compound of the invention, or a pharmaceutically acceptable salt thereof, with conventional pharmaceutical excipients. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections.

Liquid formulations may be prepared by dissolving or suspending the active substance in water or other suitable vehicles. Tablets and granules may be coated in a conventional manner.

For clinical use, the compound of the invention is formulated into pharmaceutical formulations for oral, rectal, parenteral or other mode of administration. These pharmaceutical preparations, for the intended use, are a further object of the invention.

Usually, the effective amount of active compound is between 0.1-95% by weight of the preparation, preferably between 0.2-20% by weight in preparations for parenteral use and preferably between 1 and 50% by weight in preparations for oral administration.

The dose level and frequency of dosage of the inventive compound will vary depending on a variety of factors including the potency of the inventive compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

In the preparation of pharmaceutical formulations containing the compound of the present invention in the form of dosage units for oral administration the compound may be mixed with solid, powdered ingredients, such as lactose, saccharose, sorbitol, mannitol, starch, amylopectin, cellulose derivatives, gelatine, or another suitable ingredient, as well as with disintegrating agents and lubricating agents such as magnesium stearate, calcium stearate, sodium stearyl fumarate and polyethylene glycol waxes. The mixture is then processed into granules or pressed into tablets.

Soft gelatine capsules may be prepared with capsules containing a mixture of the active compound of the invention, vegetable oil, fat, or other suitable vehicle for soft gelatine capsules. Hard gelatine capsules may contain granules of the active compound. Hard gelatine capsules may also contain the inventive compound in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance mixed with a neutral fat base; (ii) in the form of a gelatine rectal capsule which contains the active substance in a mixture with a vegetable oil, paraffin oil or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions containing from 0.2% to 20% by weight of the active ingredient and the remainder consisting of sugar or sugar alcohols and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral, e.g. intravenous, administration may be prepared as a solution of a compound of the invention in a pharmaceutically acceptable solvent, preferably in a concentration from 0.1% to 10% by weight. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The present invention includes pharmaceutical compositions comprising a therapeutically effective amount of the inventive compound for use in the treatment of a condition as defined herein, together with at least one pharmaceutically acceptable excipient, e.g. a carrier, and optionally other therapeutic and/or prophylactic ingredients.

A pharmaceutical composition according to the invention may be for topical (local) or systemic administration, e.g. for enteral administration, such as rectal or oral administration, or for parenteral administration to a mammal (especially a human), and comprises a therapeutically effective amount of the compound according to the invention or a pharmaceutically acceptable salt thereof, as active ingredient, in association with a pharmaceutically acceptable excipient, e.g. a pharmaceutically acceptable carrier. The therapeutically effective amount of the active ingredient is as defined herein above and depends e.g. on the species of mammal, the body weight, the age, the individual condition, individual pharmacokinetic data, the disease to be treated and the mode of administration.

For enteral, e.g. oral, administration, the compound of the invention may be formulated in a wide variety of dosage forms. The pharmaceutically acceptable carriers may be either solid or liquid. Solid form preparations include powders, tablets, pills, lozenges, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The formulation of the active compound may comprise an encapsulating material as carrier, providing a capsule in which the active component, with or without carriers, is surrounded by a carrier, which is in association with it.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

Exemplary compositions for rectal administration include suppositories which can contain, for example, a suitable non-irritating excipient, such as cocoa butter, synthetic glyceride esters or polyethylene glycols, which are solid at ordinary temperatures, but liquefy and/or dissolve in the rectal cavity to release the drug.

The compound of the invention also may be administered parenterally, e.g. by inhalation, injection or infusion, e.g. by intravenous, intraarterial, intraosseous, intramuscular, intracerebral, intracerebroventricular, intrasynovial, intrasternal, intrathecal, intralesional, intracranial, intracutaneous and subcutaneous injection or infusion.

Thus, for parenteral administration, the pharmaceutical composition of the invention may be in the form of a sterile injectable or infusible preparation, for example, as a sterile aqueous or oleaginous suspension. This suspension may be formulated according to techniques known in the art using suitable dispersing or wetting agents (e.g., Tween® 80), and suspending agents.

The sterile injectable or infusible preparation may also be a sterile injectable or infusible solution or suspension in a non-toxic parenterally acceptable diluent or solvent. For example, the pharmaceutical composition may be a solution in 1,3-butanediol. Other examples of acceptable vehicles and solvents that may be employed in the compositions of the present invention include, but are not limited to, mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

Solutions for parenteral use also may contain suitable stabilizing agents, and if necessary, buffer substances. Suitable stabilizing agents include antioxidizing agents, such as sodium bisulfate, sodium sulfite or ascorbic acid, either alone or combined, citric acid and its salts and sodium EDTA. Parenteral solutions may also contain preservatives, such as benzalkonium chloride, methyl- or propyl-paraben, and chlorobutanol.

For inhalation or nasal administration, suitable pharmaceutical formulations are as particles, aerosols, powders, mists or droplets, e.g. with an average size of about 10 μm in diameter or less. For example, compositions for inhalation may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions of the invention may also be administered topically, such as to the skin or to a mucous membrane. For topical application, the pharmaceutical composition may be e.g. a lotion, a gel, a paste, a tincture, a transdermal patch, a gel for transmucosal delivery.

The composition may be formulated with a suitable ointment containing the active components suspended or dissolved in a carrier. Carriers for topical administration of the compound of the invention include, but are not limited to, mineral oil, liquid petroleum, white petroleum, propylene glycol, polyoxyethylene polyoxypropylene compound, emulsifying wax and water. Alternatively, the pharmaceutical composition may be formulated as a suitable lotion or cream containing the active compound suspended or dissolved in a carrier. Suitable carriers include, but are not limited to, mineral oil, sorbitan monostearate, polysorbate 60, cetyl esters wax, cetaryl alcohol, 2-octyldodecanol, benzyl alcohol and water. The pharmaceutical compositions of this invention may also be topically applied to the lower intestinal tract by rectal suppository formulation or in a suitable enema formulation.

Suitable pharmaceutical excipients, e.g. carriers, and methods of preparing pharmaceutical dosage forms are described in Remington's Pharmaceutical Sciences, Mack Publishing Company, a standard reference text in art of drug formulation.

The pharmaceutical compositions may comprise from approximately 1% to approximately 95%, preferably from approximately 20% to approximately 90% of the compound of the invention, together with at least one pharmaceutically acceptable excipient. In general, the compound of the invention will be administered in a therapeutically effective amount by any of the accepted modes of administration for agents that serve similar utilities. Suitable daily dosages typically range from 1 to 1000 mg, e.g. 1-500 mg daily, or 1-50 mg daily, depending upon numerous factors such as the severity of the disease to be treated, the age and relative health of the patient, the potency of the compound used, the route and form of administration, and the indication towards which the administration is directed, etc. One of ordinary skill in the art of treating such diseases will be able, without undue experimentation and in reliance upon personal knowledge and the disclosure of this application, to ascertain a therapeutically effective amount of the compound of the present invention for a given disease. The compound of the invention may be administered as pharmaceutical formulations including those suitable for enteral or parenteral administration. The preferred manner of administration is generally oral using a convenient daily dosage regimen which can be adjusted, e.g. according to the degree of affliction.

The compound of the present invention may also be used or administered in combination with one or more additional therapeutically active agents. The components may be in the same formulation or in separate formulations for administration simultaneously or sequentially.

Accordingly, in a further aspect of the invention, there is provided a combination product comprising:
(A) the compound of the invention for use in the treatment of a condition as defined herein; and
(B) another therapeutic agent; whereby (A) and (B) is formulated in admixture with a pharmaceutically acceptable excipient.

Such combination products provide for the administration of the compound of the invention in conjunction with the other therapeutic agent, and may thus be presented either as separate formulations, wherein at least one of those formulations comprises the compound of the invention, and at least one comprises the other therapeutic agent, or may be presented (i.e. formulated) as a combined preparation (i.e. presented as a single formulation including the compound of the invention and the other therapeutic agent).

Thus, there is further provided:
(1) a pharmaceutical formulation including the compound of the invention for use in the treatment of a condition as defined herein, another therapeutic agent, and a pharmaceutically acceptable excipient, e.g. an adjuvant, diluent or carrier; or
(2) a kit of parts comprising, as components:
(a) a pharmaceutical formulation including the compound of the invention for use in the treatment of a condition as defined herein, in admixture with a pharmaceutically acceptable excipient, e.g. an adjuvant, diluent or carrier; and
(b) a pharmaceutical formulation including another therapeutically active agent in admixture with a pharmaceutically acceptable excipient, e.g. an adjuvant, diluent or carrier, which components (a) and (b) are each provided in a form that is suitable for administration in conjunction with the other.

In some embodiments, the compound of the invention is used in the treatment of an inflammation or an inflammatory disorder, in a combination with a further therapeutically useful compound. For example, in some embodiments, the compound of the invention is used for the treatment of an inflammatory disorder in combination with a further agent having an established activity for the same inflammatory disorder.

In some embodiments, the compound of the invention is used in the treatment of an autoimmune disorder, in a combination with a further therapeutically useful compound. For example, in some embodiments, the compound of the invention is used for the treatment of an autoimmune disorder in combination with a further agent having an established activity for the same autoimmune disorder.

In some embodiments, there is further provided:
(1) a pharmaceutical formulation including the compound of the invention for use in the treatment of a condition as hereinbefore defined, a further therapeutically active agent, and a pharmaceutically acceptable excipient, e.g. an adjuvant, diluent or carrier; or
(2) a kit of parts comprising, as components:

(a) a pharmaceutical formulation including the compound of the invention for use in the treatment of a condition as defined herein, in admixture with a pharmaceutically acceptable excipient, e.g. an adjuvant, diluent or carrier; and
(b) a pharmaceutical formulation including a further therapeutically agent, in admixture with a pharmaceutically acceptable excipient, e.g. an adjuvant, diluent or carrier, which components (a) and (b) are each provided in a form that is suitable for administration in conjunction with the other.

In some embodiments, the condition is rheumatoid arthritis and, in such embodiments, the further compound e.g. may be selected from other agents for the treatment of rheumatoid arthritis, disease-modifying anti-rheumatic drugs (DMARDs), such as e.g. methotrexate, leflunomide, hydroxychloroquine or sulfasalazine; biological treatment agents such as etanercept or infliximab; JAK inhibitors, such as tofacitinib or baricitinib. In some embodiments, the further compound also may be an analgetic, such as paracetamol, a non-steroidal anti-inflammatory drug (NSAID), such as ibuprofen, naproxen or diclofenac; or a steroid, such as prednisolone.

The components (a) and (b) in any of the above kit of parts may be administered at the same time, in sequence, or separately from each other.

As shown herein, the inventive compound has an antioxidative effect on reactive oxygen species that can contribute to the therapeutic effect in the treatment of various disorders involving intracellular reactive oxygen species, such as various inflammatory or autoimmune disorders, e.g. rheumatoid arthritis and multiple sclerosis. Therefore, in some embodiments, the inventive compound is provided in the treatment of a disorder involving intracellular reactive oxygen species, or wherein intracellular reactive oxygen species play a role in the pathogenesis, e.g. rheumatoid arthritis or multiple sclerosis.

The inventive compound further has an anti-cytokine effect that renders usefulness in the treatment of disorders where cytokines may have a pathologic effect. Therefore, in some embodiments, the inventive compound is provided for use in anti-cytokine therapy, as is useful in the treatment of a disorder where cytokines, such as TNF-α, IL10 and IL-6, have a pathogenic effect, e.g. inflammatory and/or autoimmune disorders, such as rheumatoid arthritis and multiple sclerosis.

The inventive compound 8, for use in the treatment of a disorder as mentioned herein, can be prepared by thionation of Rabeximod 3 in the presence of a thionating agent, according to the following general Reaction Scheme:

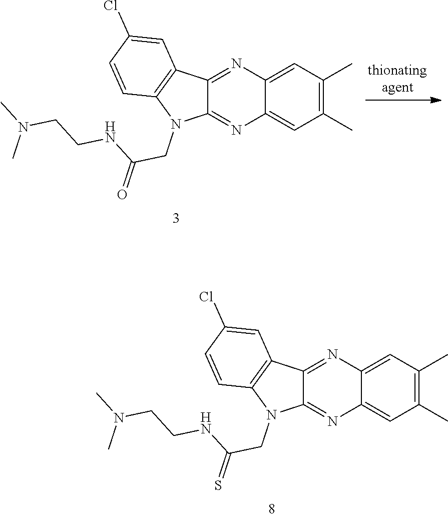

The reaction involves transforming the oxo function of Rabeximod into a thiono function (replacing the oxygen atom by a sulfur atom) by use of any suitable thionating agent, e.g. P₂S₅·2 C₅H₅N or the Lawesson reagent (CAS Number 19172-47-5). Preferably, however, P₂S₅·2 C₅H₅N is used as a thionating agent. Thionated Rabeximod thus may be prepared in a multi-step process comprising the reaction of an anion of 9-chloro-2,3-dimethyl-6H-indolo[2,3-b]quinoxaline with α-halogenated (e.g. α-brominated or α-chlorinated) methylacetate or ethylacetate, followed by condensation of obtained ester with an excess of N′,N′-dimethylethane-1,2-diamine, and thionation of the obtained product using a suitable thionating agent, such as P₂S₅·2 C₅H₅N, preferably in a liquid reaction medium comprising sulfolane, e.g. in sulfolane. The anion of 9-chloro-2,3-dimethyl-6H-indolo[2,3-b]quinoxaline may be prepared e.g. by bringing said quinoxaline in contact with a large excess of a strong base, such as potassium carbonate, in a solvent medium, such as acetone. For example, Rabeximod may be prepared in a reaction as illustrated in Reaction Scheme 1.

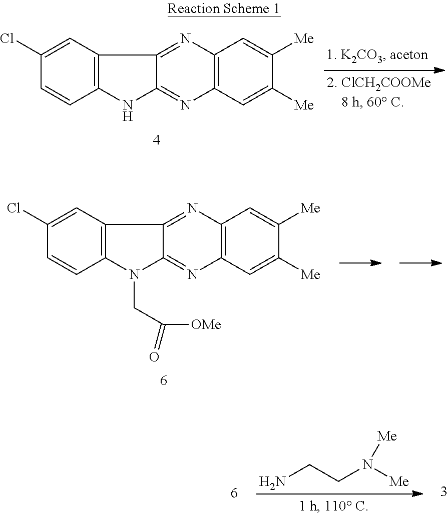 [SMILES: Cc1cc2nc3[nH]c4ccc(Cl)cc4c3nc2cc1C ; COC(=O)Cn1c2ccc(Cl)cc2c2nc3cc(C)c(C)cc3nc21 ; CN(C)CCN]

In the reaction illustrated in Reaction Scheme 1, 9-chloro-2,3-dimethyl-6H-indolo[2,3-b]quinoxaline (4) is converted to its anion with a large excess of potassium carbonate in acetone and alkylated with methyl chloroacetate to yield the ester 6. Final heating (110° C., 1 h) of 6 with 2-dimethyl-aminoethylamine gives Rabeximod 3 in a high yield. The multi-step process for preparing compound 8 comprises heating 5-chloroisatin and 4,5-dimethylphenylenediamine in an acidic medium, such as acetic acid, for a suitable time period, e.g. 8 h, whereby 2,3-dimethyl-9-chloro-6H-indolo[2,3-b]quinoxaline is obtained, which compound is subsequently used to obtain Rabeximod as described herein above, followed by thionation of Rabeximod. In some cases, thus, the multi-step process involves preparing the 2,3-dimethyl-9-chloro-6H-indolo[2,3-b]quinoxaline used as a starting material 4 in a reaction as illustrated in Reaction Scheme 2.

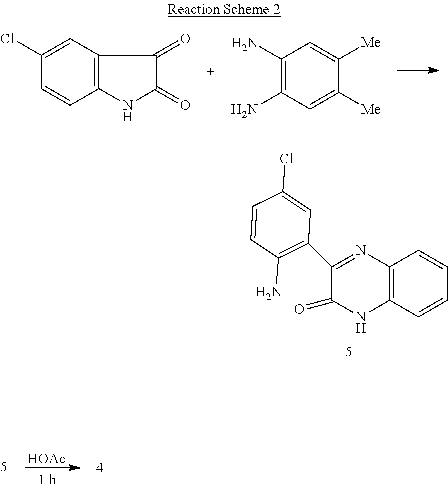 [SMILES: O=C1Nc2ccc(Cl)cc2C1=O ; Cc1cc(N)c(N)cc1C ; Nc1ccc(Cl)cc1-c1nc2ccccc2[nH]c1=O ; "HOAc"]

Thionation of Rabeximod 3 may be performed as illustrated in Reaction Scheme 3, by use of P₂S₅·2 C₅H₅N as thionating agent 7, in a solvent such as sulfolane, at 110° C. to obtain compound 8.

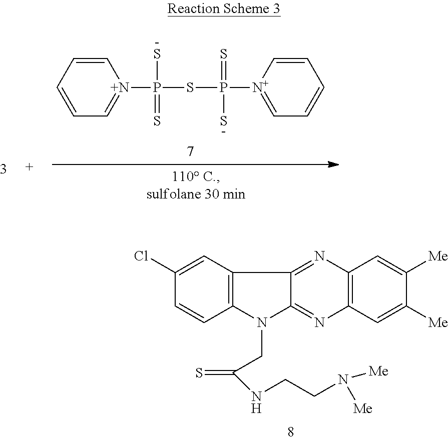 [SMILES: S=P(S)(SP(=S)(S)[n+]1ccccc1)[n+]1ccccc1 ; Cc1cc2nc3c4cc(Cl)ccc4n(CC(=S)NCCN(C)C)c3nc2cc1C]

The reactions of the above illustrated multi-step process are described in closer detail herein below.

All reactions were carried out under air atmosphere, unless otherwise indicated. All commercially available reagents were used as received without further purification. ¹H NMR and ¹³N NMR spectra were recorded on Bruker Avance/500 (C H: 500 MHz, 13 C: 125 MHz at ° C.). Melting points were measured on Mettler Toledo DSC. FT-IR spectra were recorded on a Thermo Nicolet Nexus 470 FTIR spectrometer. Reactions were monitored by High-performance liquid chromatography (HPLC) (Waters, Symmetry shield RP8, Mobile phase A: 95% acetonitrile, 5% water, 0.1% formic acid, Mobile phase A: 5% acetonitrile, 95% water, formic acid). The elemental analyses were performed by Mikroanalytisches Laboratorium Kolbe, D-46047 Oberhausen.

### 2,3-Dimethyl-9-chloro-6H-indolo[2,3-b]quinoxaline 4

A solution of 4,5-dimethylphenylenediamine (7.42 g, 0.053 mol) in acetic acid (50 mL) was added to a solution of 5-chloroisatin (10.0 g, 0.053 mol) in acetic acid (150 mL). When the two components for the synthesis of 4 were mixed in hot acetic acid a precipitate of 5 was formed within a few minutes, which slowly was converted into 4. The slurry thus formed was treated at reflux until full conversion, as determined by ¹H NMR (8 h). The mixture was allowed to cool and the solid formed was collected, washed with ethanol and dried. The crude material was recrystallized from N-methylpyrrolidone. Yield 53% (7.9 g), mp 390-393° C. IR 3030, 1621, 1594, 1460, 1446, 1325, 1285, 1211, 1164, 806, 772, 680 cm⁻¹. ¹H NMR (DMSO-d6) δ 12.08 (br.s, 1H), 8.30 (dd, J=2.2, 0.4 Hz, 1H), 8.02 (s, 1H), 7.86 (s, 1H), 7.69 (dd, J=8.6, 2.2 Hz, 1H), 7.58 (dd, J=8.6, 0.4 Hz, 1H), 2.50 (s, 6H). 13 C NMR (DMSO-d6) δ 145.8 (s), 139.6 (s), 139.2 (s), 137.7 (s), 137.5 (s), 136.2 (s), 130.4 (d), 128.0 (d), 126.6 (d), 124.7 (s), 121.1 (d), 120.5 (s), 113.5 (d), 20.0 (q), 19.7 (q). M/Z+1: 281.8.

### The Methyl Ester 6

Methyl chloroacetate (4.1 g, 37.1 mmol) was added to a slurry of 4 (10 g, 33.7 mmol) and K₂CO₃ (46.5 g, 333 mmol) in acetone (200 mL). The mixture was treated at reflux temperature until full conversion (˜8 h) according to HPLC. The slurry was allowed to cool to room temperature and poured into water. The yellow solid thus formed was collected, washed with water and dried. The crude product was used in the next step. A small sample was recrystallized from tetrahydrofuran. Yield 98% (11.9 g), mp 271-274° C. IR 2942, 1736, 1588, 1463, 1450, 1270, 1208, 877, 808, 798, 684 cm⁻¹. ¹H NMR (DMSO-d6) δ 8.36 (d, J=2.0 Hz, 1H), 8.05 (s, 1H), 7.91 (s, 1H), 7.84 (d, J=8.7 Hz, 1H), 7.78 (dd, J=8.7, 2.1 Hz, 1H), 5.41 (s, 2H), 3.71 (s, 3H), 2.51 (s, 6H). 13 C NMR (DMSO-d6) δ 168.7 (s), 144.9 (s), 142.3 (s), 140.3 (s), 138.8 (s), 138.1 (s), 137.3 (s), 136.8 (s), 130.5 (d), 128.1 (d), 126.7 (d), 125.7 (s), 121.1 (d), 120.2 (s), 112.3 (d), 52.3 (q), 42.5 (t), 20.0 (q), 19.7 (q). M/Z+1: 354.0.

### Rabeximod 3

The ester 6 (0.5 g, 1.4 mol) was added to N,N-dimethylaminoethylamine (4 mL, 17.4 mmol). After a period at reflux temperature the reaction mixture was concentrated, and water added. The solid formed was collected and washed with hot ethanol and again washed with ethanol and dried. Yield: 80% (0.45 g), mp 219-224° C. IR 3263, 2939, 2768, 1673, 1584, 1472, 1461, 1273, 1210, 868, 820, 798, 681 cm⁻¹. ¹H NMR δ 8.30 (d, 2.2 Hz, 1H), 8.24 (t, J=5.6 Hz, 1H), 7.99 (s, 1H), 7.84 (s, 1H), 7.71 (dd, J=8.7, 2.2 Hz, 1H), 7.62 (d, J=8.7 Hz, 1H), 5.10 8s, 2H), 3.16 (q, J=6.7 Hz, 2H), 2.48 (br.s, 6H), 2.28 (t, J=6.7 Hz, 2H), 2.13 (s, 6H). ¹³C NMR δ 19.6 (q), 20.0 (q), 34.3 (t), 42.6 (q), 43.9 (t), 55.6 (t), 112.2 (d), 120.2 (s), 12.8 (d), 125.3 (s), 126.4 (d), 127.9 (d), 130.1 (d), 136.2 (s), 137.2 (s), 137.8 (s), 138.6 (s), 139.7 (s), 142.5 (s), 144.9 (s), 167.5 (s). M/Z+1: 409.9.

### 2-(9-chloro-2,3-dimethyl-6H-indolo[2,3-b]quinoxalin-6-yl)-N-(2-(dimethylamino)ethyl)ethanethioamide (Thionated Rabeximod)

Rabeximod (5.6 g, 13.7 mmol) and the reagent 7 (5.2 g, 13.7 mmol) and sulfolane (45 mL) were treated at 110° C. until full conversion (˜0.5 h). The solution was allowed to cool to room temperature and poured into water (150 mL). The solid formed was collected and washed with water (50 mL), diluted ammonium hydroxide (1.25%, 100 mL) and water (50 mL) and dried. Purity: 97% (according to HPLC). Yield 92%, 5.52 g, mp 184-188° C. IR 3232, 2944, 1586, 1452, 1270, 1207, 1167, 880, 799, 685 cm⁻¹. ¹H NMR (DMSO-d6) δ (br.s, 1H), 8.31 (br.s, 1H), 8.31 (d, J=8.7, 2.1 Hz, 1H), 8.02 (s, 1H), 7.85 (s, 1H), 7.72 (dd, J=8.7, 2.1 Hz, 1H), 7.57 (d, J=8.7, 1H), 5.46 (s, 2H), 3.66 (m, 2H), 2.50 (m, 2H), 2.50 (s, 6H), 2.21 (s, 6H). ¹³C NMR (DMSO-d6) δ 196.1 (s), 145.3 (s), 139.8 (s), 138.8 (s), 138.0 (s), 137.6 (s), 130.2 (d), 128.0 (s), 126.6 (d), 125.4 (s), 120.9 (d), 120.4 (s), 112.3 (d), 55.8 (t), (t), 44.8 (q), 42.9 (t), 20.0 (q), 19.6 (q). M/Z+1: 426.0.

In Vitro Assays

Isoluminol Assay

Oxygen metabolism has an important role in the pathogenesis of both rheumatoid arthritis and multiple sclerosis. Reactive oxygen species (ROS) produced in the course of cellular oxidative phosphorylation, and by activated phagocytic cells during oxidative bursts, exceed the physiological buffering capacity and result in oxidative stress. The excessive production of ROS can damage protein, lipids, nucleic acids, and matrix components. ROS also serve as important intracellular signaling molecules that amplify the synovial inflammatory-proliferative response. Oxidative stress has been shown to induce T cell hyporesponsiveness in RA through effects on proteins and proteosomal degradation. Finally, antioxidants have been shown to ameliorate arthritis in animal models. This assay shows the ability of the tested compounds to reduce ROS.

LPS Assay

Inflammation can be induced in vitro by stimulation with LPS, manifested by the release of various cytokines. The assay shows the in vitro anti-inflammatory efficacy of the inventive compound upon LPS induced cytokine release in human whole blood. TNF-α, IL1β and IL-6 are important cytokines in the pathogenesis of RA and MS and therefore selected in this assay.

Materials and Methods

Isoluminol Assay

Frozen PBMCs, isolated from human blood (Blodcentralen, Lund, Sweden) through gradient centrifugation using Ficoll, were thawed quickly, activated with PMA at 30 ng/ml and treated with Rabeximod or the inventive compound, at different concentrations ranging from 12.5 μg/ml with 1:2 dose titrations over 8 steps. Controls included were DMSO control, PMA at 30 ng/ml and 0 μg/ml of test items. Isoluminol buffer was added to the plate (Isoluminol at 0,175 mg/ml and HRP fraction II at 1.75 U/ml in HBSS). Cell concentration was determined using a Scepter cell counter (Millipore Merck, MA, USA) and diluted at 2×10⁶ cells/ml when added to the assay plate. The luminescence signal was measured over 25 minutes.

LPS Assay

Whole blood was collected from an adult volunteer in Heparin tubes. LPS stimulation started within 60 minutes of blood draw. The blood was diluted with RPMI and then stimulated with LPS and incubated with different concentrations of either Rabeximod or the inventive compound. The samples were centrifuged and plasma harvested and analysed on Luminex to measure cytokines.

Graphs and Statistics

Graphs were performed using Prism 8 for Mac OS X (GraphPad Software, San Diego, CA, USA). Results are presented as mean values±SEM, if not otherwise stated.

Results

Isoluminol Assay

ROS production was investigated after treatment with the inventive compound or Rabeximod at different concentrations ranging from 12.5 μg/ml with 1:2 dose titrations over 8 steps. Luminescence signal was measured over 25 minutes.

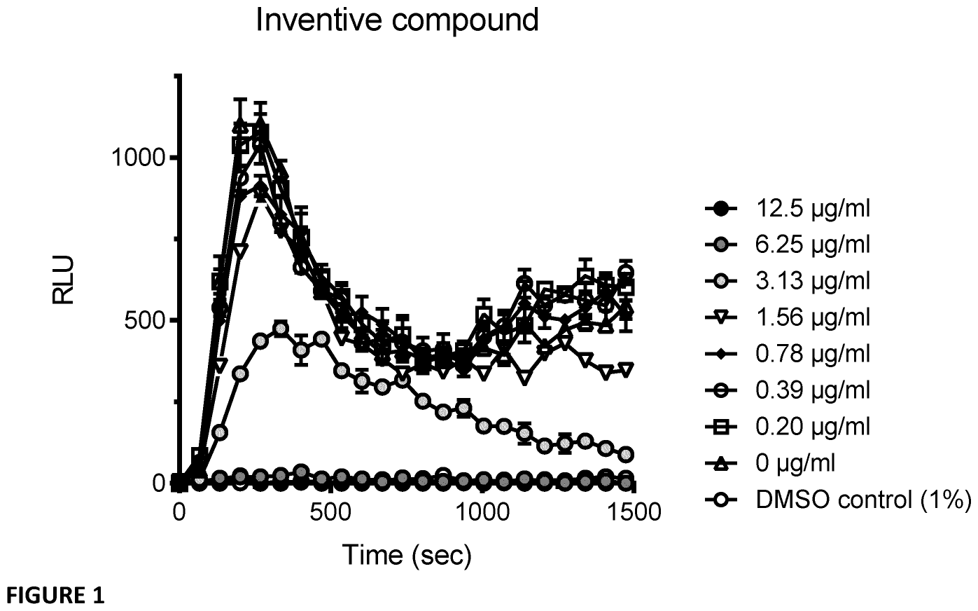

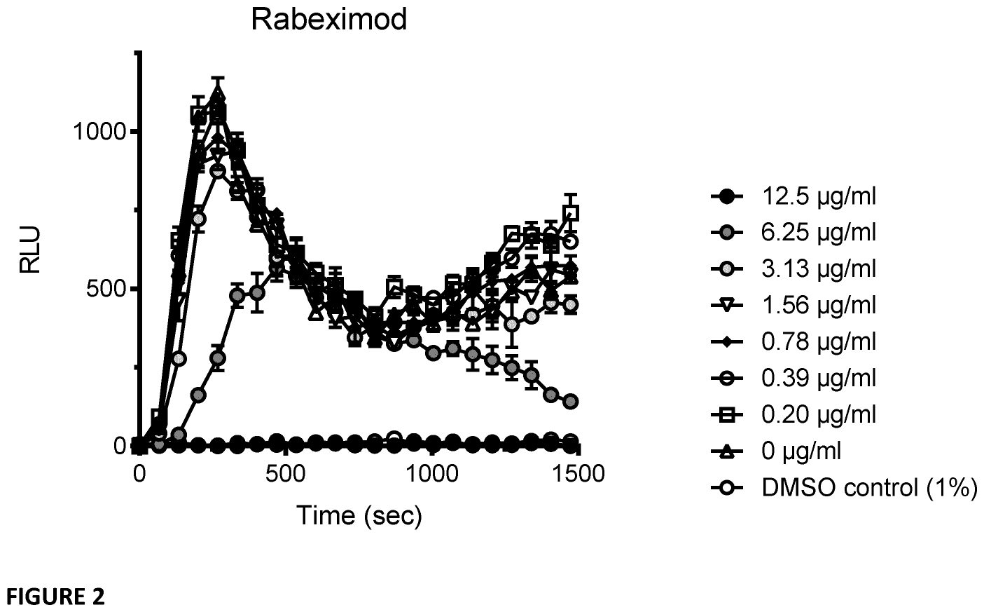

According to results in FIGS. 1 and 2, both compounds inhibit ROS production in a dose-dependent manner. The inventive compound inhibits ROS production completely from 6.25 μg/ml, whereas Rabeximod requires 12.5 μg/ml to inhibit ROS production completely.

LPS Assay

The release of cytokines and chemokines was investigated after stimulation of whole blood with LPS, and treatment with either the inventive compound or Rabeximod.

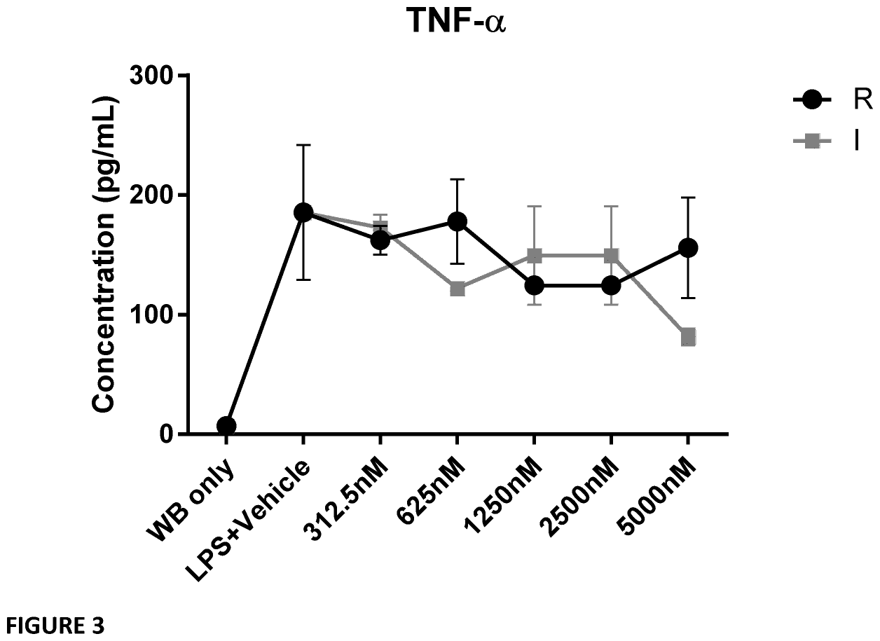

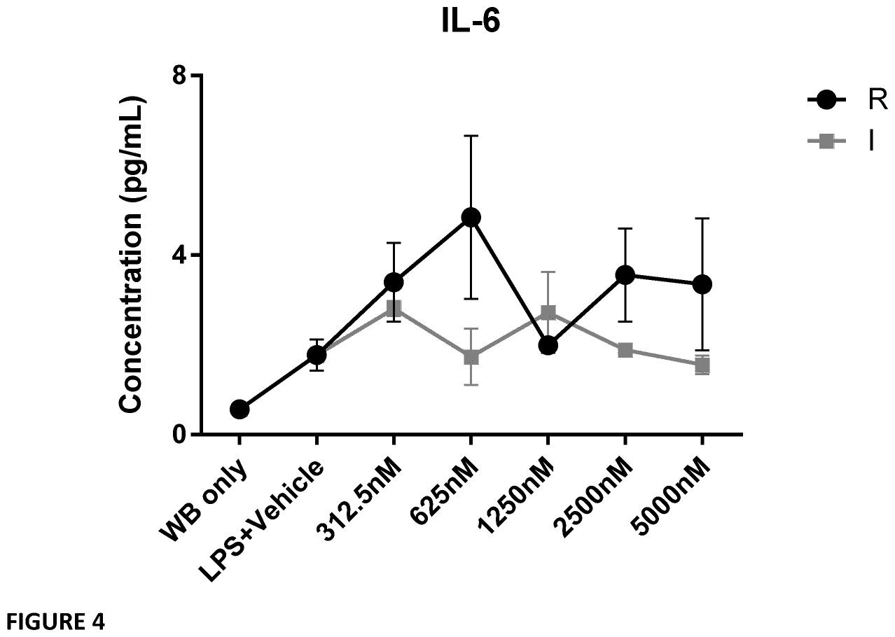

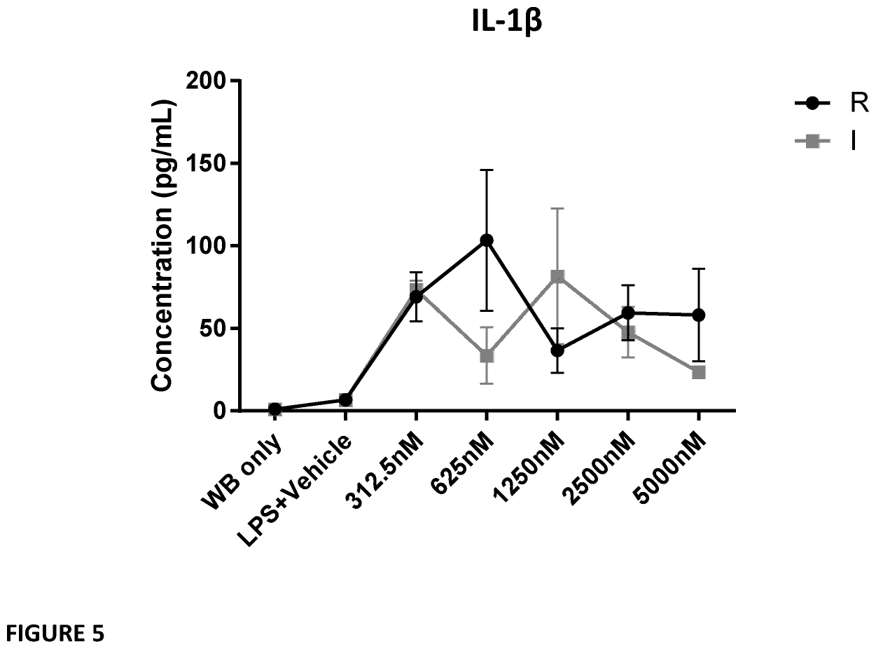

Levels of TNF-α, IL113 and IL-6 were significantly lower in samples treated with the inventive compound compared to Rabeximod at 5000 nM, with a trend of reduced cytokines levels for lower concentrations as well, see FIGS. 3-5.

In Vivo Experiments

CAIA Mouse Model

The collagen antibody induced arthritis (CAIA) arthritis in mice is a model independent of both B and T cells. CAIA is an acute model of arthritis and the disease is induced with antibodies to collagen type II. The antibodies bind to cartilage, leading to activation of the immune system followed by recruiting of macrophages and granulocytes to the joints. After an LPS boost at day five, disease progression starts and reaches a maximum around day 15.

Materials and Methods

CAIA model: Mice (DBA/1, 8-10 weeks old) were weighed at day −1 for determination of mean weight of animals in the experiment and for composition of groups. Groups were mixed within cages to avoid cage effects. The mice were injected i.v. with a cocktail of four (4) monoclonal antibodies against collagen type II (2 mg/mouse) on day 0. Day 5 the mice were injected with LPS (50 μg/mouse) i.p., in order to boost the disease. Dose preparation of test items were performed daily in corn oil. Disease was evaluated daily from day 3 in a blinded fashion using a macroscopic scoring system of the four limbs ranging from 0 to 15 (1 point for each swollen or red toe, 1 point for a swollen or red mid foot digit or knuckle, 5 points for a swollen ankle) resulting in a maximum total score of 60 for each mouse. Due to ethical restrictions, animals with a score exceeding 45 were removed from the experiment.

Immunohistochemistry: Front paws from animals, in the vehicle, Rabeximod (20 mg/kg) and the inventive compound (20 mg/kg) groups, were collected at termination of the animals. The paws were immediately transferred to 4% formaldehyde containing vials for immersion fixation. The tissues were fired for 24 hours and then transferred to 70% EtOH and stored at +4° C. until staining. Paws were decalcified, paraffin embedded, sectioned and stained for hematoxylin and eosin (H&E). Resulting slides were photographed at high resolution. Images were used for morphology analysis to study cartilage destruction and infiltration of inflammatory cells in the joints. An overall assessment for the whole paw were scored (0-3), including evaluation of synovial inflammation, synovial pannus formation and bone destruction.

Statistics

Graphs and statistical analysis were performed using Prism 8 for Mac OS X (GraphPad Software, San Diego, CA, USA). Results are presented as mean values±SEM, if not otherwise stated. Statistics were calculated using a two-tailed non-parametric Mann-Whitney test where p<0.05 was considered significant. * represents a p-value<0.05, ** represents a p-value<0.01 and *** represents a p-value<0.001.

Results

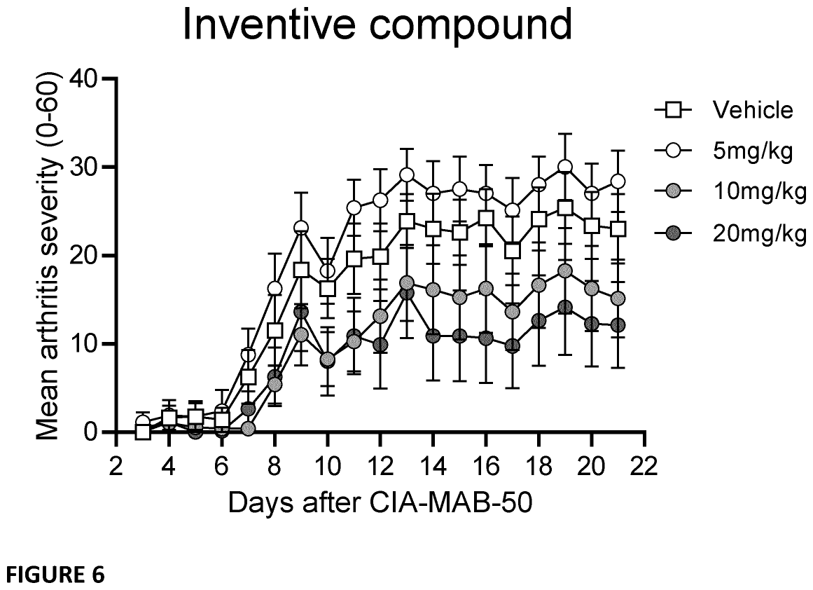

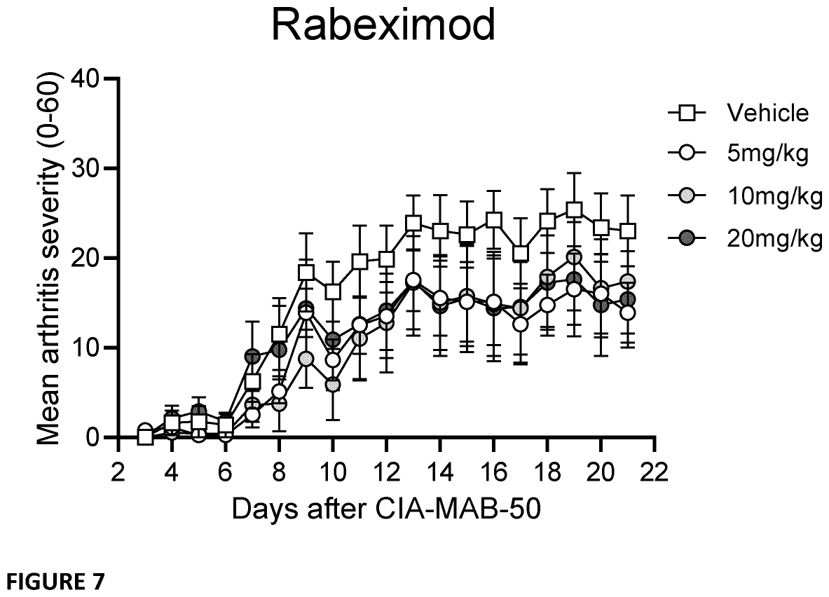

Arthritis development in mice immunized with CIA-MAB-50 and treated with vehicle (day 5, 7, 9, 11, 13 and 15), inventive compound (5, 10 and 20 mg/kg, day 5, 7, 9, 11, 13 and 15) and Rabeximod (5, 10 and 20 mg/kg, day 5, 7, 9, 11, 13 and 15). In the present CAIA study, the CAIA induced vehicle treated mice developed a moderate to severe disease with 100% incidence. The efficacy of the inventive compound and Rabeximod, respectively, at three different doses, dosed s.c. every other day starting day 5 in a total of six administrations, was evaluated. A small decrease in disease severity was seen with the inventive compound at 10 and 20 mg/kg (FIG. 6) and with Rabeximod (FIG. 7) at all three doses when comparing with vehicle, however not significant. No adverse effects from treatments were observed in animals treated with the inventive compound or Rabeximod at any of the doses tested. The data indicates a clear dose-response in mice treated with the inventive compound compared to animals treated with Rabeximod.

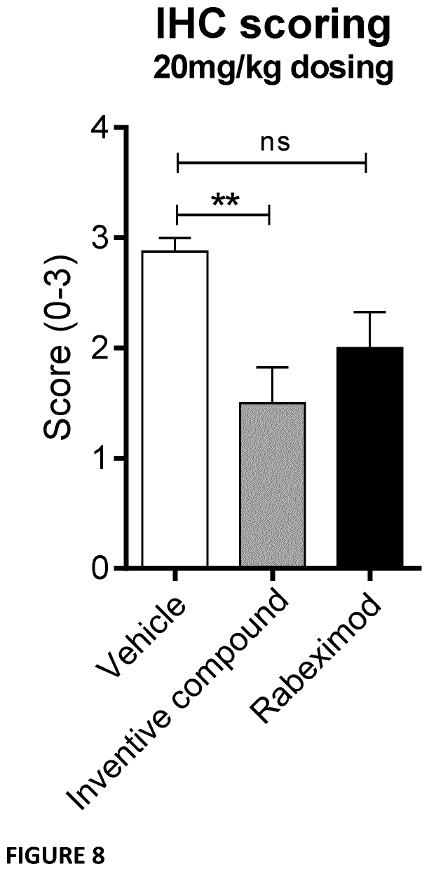

Results from IHC analysis were scored with a scoring system ranging from 0 to 3, where 0 represents a healthy tissue with no inflammation and 3 represents a massively inflamed tissue. At study termination, the inventive compound at the 20 mg/kg dose had a significantly lower histological score compared with vehicle treated animals. No significant difference was seen between vehicle and Rabeximod treated mice (FIG. 8).

PIA Rat Model

Pristane-induced arthritis (PIA) in rats is an animal model that mimics the criteria used for diagnosis of rheumatoid arthritis (RA). PIA is one of the most reproducible models of RA with high incidence and little variation in severity. Disease is characterized by a pronounced T cell dependent inflammatory response resulting in inflammation of peripheral joints. Disease is induced with a single injection of pristane and has a highly reproducible onset of arthritis between day 8-12. The most severe phase of arthritis is often between day 15-25 after induction and experiment can be terminated after 25-30 days.

Materials and Methods

For disease induction rats were anaesthetized with IsoFlo vet (3%) and oxygen. The root of the tail was cleaned with EtOH and pristane was injected subcutaneously (s.c.) (volume 200 μl) approximately 0.5 cm from the root of the tail. A slight pressure was put on the injection site for 10 seconds after injections to prevent leakage of oil. Rats were removed from anaesthetics and the breathing was monitored until recovery was ensured. Test items were administered s.c. every other day from day 5 in a total of 6 administrations (i.e. days 5, 7, 9, 11, 13 and 15) for all treatment groups, receiving Rabeximod, inventive compound or vehicle only. The compounds were formulated in corn oil and administration volume was 200 μl at a concentration of 20 mg/ml. Disease was evaluated 3 times per week in a blinded fashion from day 10 using a macroscopic scoring system of the four limbs ranging from 0 to (1 point for each swollen or red toe, 1 point for a swollen or red mid foot digit or knuckle, points for a swollen ankle) resulting in a maximum total score of 60 for each rat.

Statistics

Graphs and statistical analysis were performed using Prism 8 for Mac OS X (GraphPad Software, San Diego, CA, USA). Results are presented as mean values±SEM, if not otherwise stated.

Results

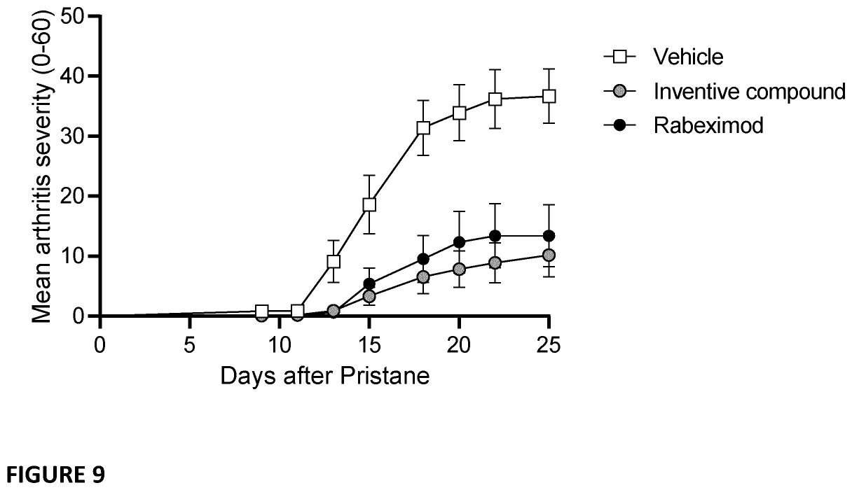

Rats treated with the inventive compound showed slightly lower RA score at all study time points measured (FIG. 9). During the most severe phase of arthritis the inventive compound had better efficacy than Rabeximod.

1.-15. (canceled)

16. A method for the treatment of a condition selected from inflammation, an inflammatory disorder, and an autoimmune disorder, by administering 2-(9-chloro-2,3-dimethyl-6H-indolo[2,3-b]quinoxalin-6-yl)-N-(2-(dimethylamino)ethyl) ethanethioamide or a pharmaceutically acceptable salt thereof, to a mammal in need of such treatment.

17. The method according to claim 16, wherein the condition is inflammation or an inflammatory disorder.

18. The method according to claim 16, wherein the condition is an autoimmune disorder.

19. The method according to claim 16, wherein the condition is rheumatoid arthritis

20. The method according to claim 16, wherein the condition is multiple sclerosis.
